SEQUENCE LISTING (84 sequences)

SEQ ID NO: 1 - protein, 237 aa, Renilla reniformis
  misc_feature (124)..(124): Xaa= Tyr or conservative substitute
MDLAKLGLKEVMPTKINLEGLVGDHAFSMEGVGEGNILEGTQEVKISVTKGAPLPFAFDIVSVAFSYGDRAYTGYPEEISDYFLQSFPEGFTYERNIRYQDGGTAIVKSDISLEDGKFIVNVEXXXXXXXXXMGPVMQQDIVGMQPSYESMYTNVTSVIGEXIIAFKLQTGIHFTYHMRTVYKSKKPVETMPLYHFIQHRLVKTNVDTASGYVVQHXXAIAAHSTIKKIEGSLPXXX

SEQ ID NO: 2 - DNA, 780 bp, Renilla reniformis
  CDS (23)..(766)
actttaagaaggagatatacatatggatctggcgaaactgggtctgaaagaagtgatgccgactaaaattaacctggaaggtctggtgggtgatcatgcgtttagcatggaaggtgtgggtgaaggtaacattctggaaggtacccaggaagtgaaaattagcgtgaccaaaggtgcgccgctgccgtttgcgtttgatattgtgagcgtggcgtttagctatggtgatcgtgcgtataccggttatccggaagaaattagcgattattttctgcagaaatttccggaaggttttacctatgaacgtggtaacattcgttatcaggatggtggtaccgcgattgtgaaaagcgatattagcctggaagatggtaaatttattgtgaacgtggaatataaaggtagcaaagacctgcgtgaaatgggtccggtgatgcagcaggatattgtgggtatgcagccgagctatgaaagcatgtataccaacgtgaccagcgtgattggtgaaggtattattgcgtttaaactgcagaccggtattcattttacctatcacatgcgtaccgtgtataaaagcaaaaaaccggtggaaaccatgccgctgtatcattttattcagcatcgtctggtgaaaaccaacgtggataccgcgagcggttatgtggtgcagcatgaaaccgcgattgcggcgcatagcaccattaaaaaaattgaaggtgcggcgcgtgaatggcgttctctcgagcaccaccaccaccaccactgagatccggctgctaa

SEQ ID NO: 3 - protein, 247 aa, Renilla reniformis
MDLAKLGLKEVMPTKINLEGLVGDHAFSMEGVGEGNILEGTQEVKISVTKGAPLPFAFDIVSVAFSYGDRAYTGYPEEISDYFLQKFPEGFTYERGNIRYQDGGTAIVKSDISLEDGKFIVNVEYKGSKDLREMGPVMQQDIVGMQPSYESMYTNVTSVIGEGIIAFKLQTGIHFTYHMRTVYKSKKPVETMPLYHFIQHRLVKTNVDTASGYVVQHETAIAAHSTIKKIEGAAREWRSLEHHHHHH

SEQ ID NO: 4 - DNA, 40 bp, Artificial Sequence
  Synthetic Sequence
actttaagaaggagatatacatatggatctggcgaaactg

SEQ ID NO: 5 - DNA, 40 bp, Artificial Sequence
  Synthetic Sequence
ggtctgaaagaagtgatgccgactaaaattaacctggaag

SEQ ID NO: 6 - DNA, 40 bp, Artificial Sequence
  Synthetic Sequence
gtctggtgggtgatcatgcgtttagcatggaaggtgtggg

SEQ ID NO: 7 - DNA, 40 bp, Artificial Sequence
  Synthetic Sequence
tgaaggtaacattctggaaggtacccaggaagtgaaaatt

SEQ ID NO: 8 - DNA, 40 bp, Artificial Sequence
  Synthetic Sequence
agcgtgaccaaaggtgcgccgctgccgtttgcgtttgata

SEQ ID NO: 9 - DNA, 40 bp, Artificial Sequence
  Synthetic Sequence
ttgtgagcgtggcgtttagctatggtgatcgtgcgtatac

SEQ ID NO: 10 - DNA, 40 bp, Artificial Sequence
  Synthetic Sequence
cggttatccggaagaaattagcgattattttctgcagaaa

SEQ ID NO: 11 - DNA, 40 bp, Artificial Sequence
  Synthetic Sequence
tttccggaaggttttacctatgaacgtggtaacattcgtt

SEQ ID NO: 12 - DNA, 40 bp, Artificial Sequence
  Synthetic Sequence
atcaggatggtggtaccgcgattgtgaaaagcgatattag

SEQ ID NO: 13 - DNA, 40 bp, Artificial Sequence
  Synthetic Sequence
cctggaagatggtaaatttattgtgaacgtggaatataaa

SEQ ID NO: 14 - DNA, 40 bp, Artificial Sequence
  Synthetic Sequence
ggtagcaaagacctgcgtgaaatgggtccggtgatgcagc

SEQ ID NO: 15 - DNA, 40 bp, Artificial Sequence
  Synthetic Sequence
aggatattgtgggtatgcagccgagctatgaaagcatgta

SEQ ID NO: 16 - DNA, 40 bp, Artificial Sequence
  Synthetic Sequence
taccaacgtgaccagcgtgattggtgaaggtattattgcg

SEQ ID NO: 17 - DNA, 40 bp, Artificial Sequence
  Synthetic Sequence
tttaaactgcagaccggtattcattttacctatcacatgc

SEQ ID NO: 18 - DNA, 40 bp, Artificial Sequence
  Synthetic Sequence
gtaccgtgtataaaagcaaaaaaccggtggaaaccatgcc

SEQ ID NO: 19 - DNA, 40 bp, Artificial Sequence
  Synthetic Sequence
gctgtatcattttattcagcatcgtctggtgaaaaccaac

SEQ ID NO: 20 - DNA, 40 bp, Artificial Sequence
  Synthetic Sequence
gtggataccgcgagcggttatgtggtgcagcatgaaaccg

SEQ ID NO: 21 - DNA, 40 bp, Artificial Sequence
  Synthetic Sequence
cgattgcggcgcatagcaccattaaaaaaattgaaggtgc

SEQ ID NO: 22 - DNA, 40 bp, Artificial Sequence
  Synthetic Sequence
ggcgcgtgaatggcgttctctcgagcaccaccaccaccac

SEQ ID NO: 23 - DNA, 40 bp, Artificial Sequence
  Synthetic Sequence
gtggtggtggtggtgctcgagagaacgccattcacgcgcc

SEQ ID NO: 24 - DNA, 40 bp, Artificial Sequence
  Synthetic Sequence
gcaccttcaatttttttaatggtgctatgcgccgcaatcg

SEQ ID NO: 25 - DNA, 40 bp, Artificial Sequence
  Synthetic Sequence
cggtttcatgctgcaccacataaccgctcgcggtatccac

SEQ ID NO: 26 - DNA, 40 bp, Artificial Sequence
  Synthetic Sequence
gttggttttcaccagacgatgctgaataaaatgatacagc

SEQ ID NO: 27 - DNA, 40 bp, Artificial Sequence
  Synthetic Sequence
ggcatggtttccaccggttttttgcttttatacacggtac

SEQ ID NO: 28 - DNA, 40 bp, Artificial Sequence
  Synthetic Sequence
gcatgtgataggtaaaatgaataccggtctgcagtttaaa

SEQ ID NO: 29 - DNA, 40 bp, Artificial Sequence
  Synthetic Sequence
cgcaataataccttcaccaatcacgctggtcacgttggta

SEQ ID NO: 30 - DNA, 40 bp, Artificial Sequence
  Synthetic Sequence
tacatgctttcatagctcggctgcatacccacaatatcct

SEQ ID NO: 31 - DNA, 40 bp, Artificial Sequence
  Synthetic Sequence
gctgcatcaccggacccatttcacgcaggtctttgctacc

SEQ ID NO: 32 - DNA, 40 bp, Artificial Sequence
  Synthetic Sequence
tttatattccacgttcacaataaatttaccatcttccagg

SEQ ID NO: 33 - DNA, 40 bp, Artificial Sequence
  Synthetic Sequence
ctaatatcgcttttcacaatcgcggtaccaccatcctgat

SEQ ID NO: 34 - DNA, 40 bp, Artificial Sequence
  Synthetic Sequence
aacgaatgttaccacgttcataggtaaaaccttccggaaa

SEQ ID NO: 35 - DNA, 40 bp, Artificial Sequence
  Synthetic Sequence
tttctgcagaaaataatcgctaatttcttccggataaccg

SEQ ID NO: 36 - DNA, 40 bp, Artificial Sequence
  Synthetic Sequence
gtatacgcacgatcaccatagctaaacgccacgctcacaa

SEQ ID NO: 37 - DNA, 40 bp, Artificial Sequence
  Synthetic Sequence
tatcaaacgcaaacggcagcggcgcacctttggtcacgct

SEQ ID NO: 38 - DNA, 40 bp, Artificial Sequence
  Synthetic Sequence
aattttcacttcctgggtaccttccagaatgttaccttca

SEQ ID NO: 39 - DNA, 40 bp, Artificial Sequence
  Synthetic Sequence
cccacaccttccatgctaaacgcatgatcacccaccagac

SEQ ID NO: 40 - DNA, 40 bp, Artificial Sequence
  Synthetic Sequence
cttccaggttaattttagtcggcatcacttctttcagacc

SEQ ID NO: 41 - DNA, 40 bp, Artificial Sequence
  Synthetic Sequence
cagtttcgccagatccatatgtatatctccttcttaaagt

SEQ ID NO: 42 - DNA, 25 bp, Artificial Sequence
  Synthetic Sequence
cgcagtcgaccgttttttttttttt

SEQ ID NO: 43 - DNA, 23 bp, Artificial Sequence
  Synthetic Sequence
aagcagtggtatcaacgcagagt

SEQ ID NO: 44 - DNA, 27 bp, Artificial Sequence
  Synthetic Sequence
gatatacatatgggtccggtgatgcag

SEQ ID NO: 45 - DNA, 27 bp, Artificial Sequence
  Synthetic Sequence
gatatacatatgtctgatatttcatta

SEQ ID NO: 46 - protein, 237 aa, Renilla reniformis
  misc_feature (66)..(66): Xaa = Ser or Gln or conservative substitution
MDLAKLGLKEVMPTKINLEGLVGDHAFSMEGVGEGNILEGTQEVKISVTKGAPLPFAFDIVSVAFXYGXRAYTGYPEEISDYFLQSFPEGFTYERNIRYQDGGTAIVKSDISLEDGKFIVNVDFKGNKDLRRMGPVMQQDIVGMQPSYESMYTNVTSVIGECIIAFKLQTGKHFTYHMRTVYKSKKPVETMPLYHFIQHRLVKTNVDTASGYVVQHETAIAAHSTIKKIEGSLPXXX

SEQ ID NO: 47 - DNA, 40 bp, Artificial Sequence; identical to SEQ ID NO: 4
  Upper Primer 1

SEQ ID NO: 48 - DNA, 40 bp, Artificial Sequence; identical to SEQ ID NO: 5
  Upper Primer 2

SEQ ID NO: 49 - DNA, 40 bp, Artificial Sequence; identical to SEQ ID NO: 6
  Upper Primer 3

SEQ ID NO: 50 - DNA, 40 bp, Artificial Sequence; identical to SEQ ID NO: 7
  Upper Primer 4

SEQ ID NO: 51 - DNA, 40 bp, Artificial Sequence; identical to SEQ ID NO: 8
  Upper Primer 5

SEQ ID NO: 52 - DNA, 40 bp, Artificial Sequence
  Upper Primer 6
ttgtgaacgtggcgtttcagtatggtaaccgtgcgtatac

SEQ ID NO: 53 - DNA, 40 bp, Artificial Sequence
  Upper Primer 7
cggttatccggaagaaattagcgattattttctgcagagc

SEQ ID NO: 54 - DNA, 37 bp, Artificial Sequence
  Upper Primer 8
tttccggaaggttttacctatgaacgtaacattcgtt

SEQ ID NO: 55 - DNA, 40 bp, Artificial Sequence; identical to SEQ ID NO: 12
  Upper Primer 9

SEQ ID NO: 56 - DNA, 40 bp, Artificial Sequence
  Upper Primer 10
cctggaagatggtaaatttattgtgaacgtggattttaaa

SEQ ID NO: 57 - DNA, 37 bp, Artificial Sequence
  Upper Primer 11
ggtagcgacctgcgtcgtatgggtccggtgatgcagc

SEQ ID NO: 58 - DNA, 40 bp, Artificial Sequence; identical to SEQ ID NO: 15
  Upper Primer 12

SEQ ID NO: 59 - DNA, 40 bp, Artificial Sequence
  Upper Primer 13
taccaacgtgaccagcgtgattggtgaatgcattattgcg

SEQ ID NO: 60 - DNA, 40 bp, Artificial Sequence
  Upper Primer 14
tttaaactgcagaccggtaaacattttacctatcacatgc

SEQ ID NO: 61 - DNA, 40 bp, Artificial Sequence; identical to SEQ ID NO: 18
  Upper Primer 15

SEQ ID NO: 62 - DNA, 40 bp, Artificial Sequence; identical to SEQ ID NO: 19
  Upper Primer 16

SEQ ID NO: 63 - DNA, 40 bp, Artificial Sequence; identical to SEQ ID NO: 20
  Upper Primer 17

SEQ ID NO: 64 - DNA, 40 bp, Artificial Sequence
  Upper Primer 18
cgattgcggcgcatagcaccattaaaaaaattgaaggtag

SEQ ID NO: 65 - DNA, 40 bp, Artificial Sequence
  Upper Primer 19
cctgccggaatgggtgtctctcgagcaccaccaccaccac

SEQ ID NO: 66 - DNA, 40 bp, Artificial Sequence
  Lower Primer 1
ttgttagcagccggatctcagtggtggtggtggtgctcga

SEQ ID NO: 67 - DNA, 40 bp, Artificial Sequence
  Lower Primer 2
gagacacccattccggcaggctaccttcaatttttttaat

SEQ ID NO: 68 - DNA, 40 bp, Artificial Sequence
  Lower Primer 3
ggtgctatgcgccgcaatcgcggtttcatgctgcaccaca

SEQ ID NO: 69 - DNA, 40 bp, Artificial Sequence
  Lower Primer 4
taaccgctcgcggtatccacgttggttttcaccagacgat

SEQ ID NO: 70 - DNA, 40 bp, Artificial Sequence
  Lower Primer 5
gctgaataaaatgatacagcggcatggtttccaccggttt

SEQ ID NO: 71 - DNA, 40 bp, Artificial Sequence
  Lower Primer 6
tttgcttttatacacggtacgcatgtgataggtaaaatgt

SEQ ID NO: 72 - DNA, 40 bp, Artificial Sequence
  Lower Primer 7
ttaccggtctgcagtttaaacgcaataatgcattcaccaa

SEQ ID NO: 73 - DNA, 40 bp, Artificial Sequence
  Lower Primer 8
tcacgctggtcacgttggtatacatgctttcatagctcgg

SEQ ID NO: 74 - DNA, 37 bp, Artificial Sequence
  Lower Primer 9
ctgcatacccacaatatcctgctgcatcaccggaccc

SEQ ID NO: 75 - DNA, 40 bp, Artificial Sequence
  Lower Primer 10
atacgacgcaggtcgctacctttaaaatccacgttcacaa

SEQ ID NO: 76 - DNA, 40 bp, Artificial Sequence
  Lower Primer 11
taaatttaccatcttccaggctaatatcgcttttcacaat

SEQ ID NO: 77 - DNA, 37 bp, Artificial Sequence
  Lower Primer 12
cgcggtaccaccatcctgataacgaatgttacgttca

SEQ ID NO: 78 - DNA, 40 bp, Artificial Sequence
  Lower Primer 13
taggtaaaaccttccggaaagctctgcagaaaataatcgc

SEQ ID NO: 79 - DNA, 40 bp, Artificial Sequence
  Lower Primer 14
taatttcttccggataaccggtatacgcacggttaccata

SEQ ID NO: 80 - DNA, 40 bp, Artificial Sequence
  Lower Primer 15
ctgaaacgccacgttcacaatatcaaacgcaaacggcagc

SEQ ID NO: 81 - DNA, 40 bp, Artificial Sequence
  Lower Primer 16
ggcgcacctttggtcacgctaattttcacttcctgggtac

SEQ ID NO: 82 - DNA, 40 bp, Artificial Sequence
  Lower Primer 17
cttccagaatgttaccttcacccacaccttccatgctaaa

SEQ ID NO: 83 - DNA, 40 bp, Artificial Sequence
  Lower Point 18
cgcatgatcacccaccagaccttccaggttaattttagtc

SEQ ID NO: 84 - DNA, 40 bp, Artificial Sequence
  Lower Primer 19
ggcatcacttctttcagacccagtttcgccagatccatat

(19) United States
(12) Patent Application Publication
(10) Pub. No.: US 2003/0013849 A1
(43) Pub. Date: Jan. 16, 2003

(54) Renilla reniformis green fluorescent protein

(76) Inventors: William W. Ward, Metuchen, NJ (US); Catherine Thomson, Edinburgh (GB)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/135,965

(22) Filed: Apr. 30, 2002

Related U.S. Application Data
(60) Provisional application No. 60/162,584, filed on Oct. 29, 1999.
(60) Provisional application No. 60/213,093, filed on Jun. 21, 2000.
(60) Provisional application No. 60/223,805, filed on Aug. 8, 2000.
(60) Provisional application No. 60/287,611, filed on Apr. 30, 2001.

(51) Int. Cl. C07H 21/04; C12P 21/04; C07K 14/435; C12P 21/02; C12N 5/06

(52) U.S. Cl. 530/350; 435/69.7; 435/320.1; 435/325; 536/23.5

(57) ABSTRACT

Green fluorescent protein (GFP) polypeptides from Renilla reniformis and Renilla kollikeri are disclosed. The amino acid sequence of R. reniformis GFP and back-translated nucleotide sequences of nucleic acids encoding the R. reniformis GFP are also disclosed. These isolated polypeptides, along with the pertinent amino acid and nucleotide sequence information, are useful in a variety of applications for which GFPs from other sources (e.g., Aequoria) are currently employed. Techniques for using the Renilla GFPs are disclosed, along with advantages of Renilla GFP as compared with currently available GFPs.

This application is a continuation-in-part of co-pending U.S. Application No. [not yet assigned], which is a national filing under 35 U.S.C. §371 of International Application No. PCT US00/29976, filed Oct. 30, 2000, which claims benefit of U.S. Provisional Application Nos. 60/162,584, filed Oct. 29, 1999, 60/213,093, filed Jun. 21, 2000 and 60/223,805, filed Aug. 8, 2000. This application also claims benefit of U.S. Provisional Application No. 60/287,611, filed Apr. 30, 2001. Each of the aforementioned patent applications is incorporated by reference herein in its entirety.

Pursuant to 35 U.S.C. §202(c), it is acknowledged that the U.S. Government has certain rights in the invention described herein, which was made in part with funds from a National Science Foundation-Advanced Technological Education grant (DUE# 9602356).

### FIELD OF THE INVENTION

This invention relates to the field of biotechnology research products, fluorescent proteins, fluorescence microscopy, high throughput screening, diagnostics, and the monitoring by fluorimetric remote sensing of agricultural and environmental acreage. In particular, this invention provides a isolated or synthetic green fluorescent protein (GFP), having amino acid sequence and functional features of the GFP from Renilla reniformis and Renilla kollikeri and natural or synthetic genes that encode Renilla GFPs.

### BACKGROUND OF THE INVENTION

Various scientific and scholarly articles are referred to in parentheses throughout the specification. These articles are incorporated by reference herein to describe the state of the art to which this invention pertains.

Many species of coelenterates jellyfish, hydroids, sea pansies, and sea pens) are bioluminescent. A rise in the intracellular concentration of calcium causes the oxidation of a protein-bound luciferin molecule, resulting in formation of excited-state oxyluciferin. The oxyluciferin may emit blue light by direct de-excitation or may transfer the energy by a radiationless mechanism to the non-catalytic accessory protein, the green fluorescent protein (GFP), which subsequently emits green light.

Thus, GFP acts to shift the color of bioluminescence from blue to green in luminous coelenterates and to increase the quantum yield of light emission (Ward and Cormier, 1979, J. Biol. Chem. 254:781-788). Nearly all naturally occurring GFPs emit light with wavelength maxima in the 490-520 nm range, with most centered at 508-509 nm. The range of excitation maxima is however much broader, 395-498 nm (Ward, 1998, In Green Fluorescent Protein: Properties, Applications and Protocols, pp 45-75, ed. M. Chalfie and S. Kain, Wiley-Liss).

The jellyfish, Aequorea victoria, produces bioluminescence that is typical of the hydrozoan family of coelenterates. The A. victoria GFP is the best characterized of the GFPs. The gene for GFP was first isolated from Aequorea (Prasher et al., 1992, Gene 111:229-233) and later demonstrated capable of functional expression as a transgene (Chalfie et al., 1994, Science 263:802-805).

The isolation of the Aequorea GFP gene has led to a proliferation of GFP mutants and ever-increasing numbers of GFP applications. Key to the usefulness of this gene is that it needs no added substrates or cofactors (other than those factors found in typical in vitro translation reagents) to produce a functional gene product. It can be readily expressed in heterologous organisms. GFP as produced, fluoresces: it can shift the color of experimentally introduced blue or ultra-violet light to an emitted green light. It is therefore useful as a non-invasive marker in living cells, enabling applications such as cell lineage tracing, reporter gene expression, and measurement of protein-protein interactions.

Fluorescent GFP has been expressed as a functional transgene in a wide range of cells and/or organisms, including bacteria, yeast, slime mold, plants, Drosophila, zebra fish and mammalian cells. GFP can function as a useful protein tag because it tolerates C-terminal and N-terminal fusion to a broad range of proteins without loss of its fluorescent properties. Wild-type GFP is typically distributed in the cytoplasm and nucleus of heterologous cells in which it is expressed, but it can also be targeted to the nucleus, mitochondria, chloroplasts, secretory pathways, plasma membrane or cytoskeleton by GFP gene fusions with sequences encoding specific targeting or with coding sequences of entire proteins.

Aequorea GFP is composed of 238 amino acids which provide a polypeptide size of approximately 27 kDa. It is the only known GFP molecule that has an excitation maximum in the ultraviolet region, with its major excitation peak at 395 nm and a minor excitation peak at 475 nm. Its emission peak is at 508 nm. Conventional protein sequencing and gene sequencing of a wide variety of Aequorea GFP mutants as well as X-ray crystallography have lead to the identification of the chromophore, derived from residues 64-69 of the primary amino acid sequence (Yang et al., 1996, Nature Biotechnology 14:1246-1251; Ward 1998, supra). Post-translational modifications of the protein result in a cyclized tripeptide originating from these residues. No other enzymes or cofactors are required for the cyclization of the apoprotein, however molecular oxygen is clearly required. Natural and induced mutations in the amino acid sequence of Aequorea GFP lead to shifts in the absorbance spectrum, enhancements in fluorescence, and increases in temperature tolerance (Yang et al., 1996, supra).

Several variants and mutants of the Aequorea GFP have been discovered and developed. Some of these variants (especially those with variations in and around the chromophore) are known to have physical properties that are advantageous in specific situations. These variations in Aequorea GFP are well known in the art (Yang et al., 1996, supra).

The GFP from the anthozoan coelenterates Renilla reniformis and Renilla kollikeri, the sea pansies, has many functional advantages over the Aequorea GFP. While its emission spectrum is very similar to Aequorea GFP (wavelength max=509 nm), the excitation (or absorption) spectrum of Renilla GFP is very different. Renilla GFP has excitation peaks at 498 nm and 470 nm, with a half band width of approximately 15 nm at both. In contrast, Aequorea GFP has excitation peaks at 393 nm and 473 nm, with a half band width of approximately 30 nm at both (Ward et al., 1980, Photochem. Photobiol. 31:611-615). The Renilla GFP absorbs very little between 320-390 nm, where Aequorea GFP has considerable absorption. This region of low absorption is a strong asset to many applications related to fluorescence microscopy where the 320-390 nm range could be used to excite a second “reporter” chromophore, such as DAPI, while the higher wavelength is used to excite the Renilla GFP. The transparent window (320nm-390 nm) in Renilla reniformis and Renilla kollikeri GFP excitation also facilitates mathematical noise subtraction in high throughput screening and in remote sensing applications where multiwavelength excitation is employed.

Renilla GFP also has a much higher extinction coefficient, 133,000 L * mol⁻¹ * cm⁻¹ at 498 nm as compared to 27,600 L * mol⁻¹ * cm⁻¹ at 397 nm for Aequorea GFP, while they both have similar quantum yields of 0.80. This higher extinction coefficient is a great benefit to all uses of GFP, but particularly so in application for in vivo expression in such diverse fields as high throughput screening, diagnostics, and the remote fluorimetric monitoring of agricultural and environmental change. The Aequorea GFP has proved adequate when expressed by a strong promoter, but often inadequate when fused to a weaker promoter. Many applications that seek to characterize the in vivo regulation of a weaker promoter need a “brighter” GFP in order to succeed. Moreover, the higher stability of Renilla GFP when subjected to pH extremes, detergents and chaotropic agents has general advantages in many in vitro applications such as fixation of tissue and diagnostic kits.

While a great deal is known about the physical properties of Renilla GFP, little is known about its amino acid sequence or the nucleic acid sequence of its gene, presumably due to one or more factors including: (1) difficulty in obtaining the organism, (2) difficulty and complexity of purifying GFP from Renilla, and (3) difficulty in obtaining suitable DNA or RNA for cloning purposes. The GFP purified directly from Renilla is currently too costly to sell commercially and, in any event, tends to consist of a heterogeneous population, possibly the result of multiple GFP genes in the natural population or limited C-terminal truncation of the gene product as occurs in native Aequorea GFP.

Having the complete sequence of the Renilla reniformis or R. kollikeri GFP would put this tool within the reach of the biotechnology community for cloning, expression and diagnostic and other applications. The six amino acid residues corresponding to the chromophore region of Renilla GFP have been identified (San Pietro et al., 1993, Photochem. Photobiol. 57:63s), but this information is hardly enough to synthesize a protein with all the unique properties of Renilla GFP or to isolate native nucleic acids that encode it. Making the Renilla GFP protein and nucleic acids available would enable a new range of GFP applications.

### SUMMARY OF THE INVENTION

In accordance with the present invention, the amino acid sequence of Renilla reniformis GFP has now been determined. From this information, it is now possible to produce a synthetic GFP having the defining characteristics of R. reniformis GFP. It is also possible to design and produce nucleic acid molecules encoding the Renilla reniformis GFP.

According to one aspect of the invention, a synthetic green fluorescent protein (GFP) is provided. This protein has the sequence of the Renilla GFP set forth in SEQ ID NO: 1 or SEQ ID NO: 46. The synthetic GFP of the invention has excitation peaks at 470 nm and 498 nm, and an emission peak at 509 nm, and a transparent absorbance window from 320-390 nm. The synthetic Renilla GFP also has a very high molar extinction coefficient, 133,000 at 498 nm, making it ideal for applications where the current standard Aequorea GFP is not intense enough. Additionally, the Renilla GFP is stable at high and low pH extremes, in 8 M urea, 6 M guanidine hydrochloride and 1% SDS. Because of its transparent absorbance window from 320 nm to 390 nm, the synthetic Renilla GFP is better suited than Aequorea GFP for techniques involving double fluorescent-labeling. In addition, the transparent absorption window that exists in Renilla GFP provides a mechanism of noise suppression (removal of autofluorescence and scatter) with the use of polychromatic excitation. The broader stability range also allows the synthetic Renilla GFP to be used in applications where Aequorea GFP would lose fluorescence signal.

According to a second aspect of the invention, a nucleic acid molecule that encodes Renilla GFP is provided. In a preferred embodiment, the nucleic acid encodes the protein sequence defined in SEQ ID NO: 1 or SEQ ID NO: 46. In another preferred embodiment, the nucleic acid encodes the amino acid sequence of SEQ ID NO: 1 or SEQ ID NO: 46 and is isolated from Renilla. In another preferred embodiment, the nucleic acid encodes the amino acid sequence of SEQ ID NO: 1 or SEQ ID NO: 46 using optimized mammalian or prokaryotic codon usage.

Also provided in accordance with the present invention are standard GFPs. Such standards are useful in order to allow calibration of many fluorescence-based biological assays as well the fluorescence measuring instruments. These standards are also provided as kits for ease of use, wherein standard concentrations or dilutions are provided, along with certification of the standard properties and biophysical parameters, and instructions for use. A method for the use of such standards in calibrating instruments and fluorescence-based assays is further provided.

Further provided in the present invention are antibodies to the GFPs of the invention. These antibodies are useful for a variety of purposes; they are particularly of use in purification and characterization of the GFPs and variants thereof. In addition to the antibodies to the GFP, the instant invention includes antibodies which are fused to or tagged by a GFP molecule. These antibodies, which still retain their useful binding characteristics are readily detected as they also provide the fluorescent properties of the GFP. Such antibodies further include genetically-designed antibody fragments which can be expressed and purified. Typically these are produced from a gene construct which includes a sequence encoding a heavy chain, or binding fragment of an immunoglobulin molecule fused in-frame with a GFP-encoding sequence. Such immuno-GFP molecules are useful for a variety of purposes including hybrid assays with the specificity of immunoassays and the improved detection of GFP fluorescent assays. The use of GFPs in this capacity also provides for use of multiple fluorescent tags within the immunoassays.

A method for the reduction of background noise in fluorescence-based biological assays is also provided. This method is facilitated by the window of low absorbance in the GFP of the present invention. Other GFPs lack a window of low absorbance from 320 nm through 390 nm, whereas the Renilla GFPs of the instant invention have near-transparent window of absorption in this range. This can be utilized to reduce background significantly and to greatly increase the signal-to-noise ratio, allowing more sensitive detection in biological assays based on fluorescence detection.

Other features and advantages of the present invention will be better understood by reference to the figure and detailed description that follow.

### BRIEF DESCRIPTION OF THE DRAWINGS

FIG. 1. Absorption spectrum of Renilla kollikeri GFP.

### DETAILED DESCRIPTION OF THE INVENTION

I. Definitions

Various terms relating to the biological molecules of the present invention are used throughout the specifications and claims.

Where used herein, “isolated” means altered “by the hand of man” from the natural state. If a composition or substance occurs in nature, it has been “isolated” for example, when changed or removed from its original environment. For example, a polynucleotide or a polypeptide naturally present in a living animal is not “isolated,” but the same polynucleotide or polypeptide separated from the coexisting materials of its natural state, or present through synthetic means, is “isolated”, as the term is employed herein.

With reference to nucleic acids of the invention, the term “isolated nucleic acid” is sometimes used. This term, when applied to genomic DNA, refers to a DNA molecule that is separated from sequences with which it is immediately contiguous (in the 5′ and 3′ directions) in the naturally-occurring genome of the organism from which it was derived. For example, the “isolated nucleic acid” may comprise a DNA molecule inserted into a vector, such as a plasmid or virus vector, or integrated into the genomic DNA of a procaryote or eukaryote. An “isolated nucleic acid molecule” may also comprise a cDNA molecule or a synthesized nucleic acid molecule. An “isolated nucleic acid” also may be a synthetic nucleic acid.

With respect to RNA molecules of the invention the term “isolated nucleic acid” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form (the term “substantially pure” is defined below). Alternatively, an entire class of RNA molecules is sometimes deemed “isolated” when is separated from other biomolecules and/or other classes of RNA (e.g. tRNA and rRNA). For example, the class of polyadenylated RNA is often isolated in order to clone cDNA from a specific messenger RNA.

With respect to protein, the term “isolated protein” or “isolated and purified protein” is sometimes used herein. This term often refers to a protein which has been sufficiently separated from other proteins with which it would naturally be associated, so as to exist in “substantially pure” form. Alternatively, this term may refer to a protein produced by expression of an isolated nucleic acid molecule of the invention. An “isolated protein” also may be a synthetic polypeptide comprising naturally occurring or non-naturally occurring amino acid residues.

The term “polynucleotide” generally refers to any polyribonucleotide or polydeoxribonucleotide, which may be unmodified RNA or DNA or modified RNA or DNA. “Polynucleotides” include, without limitation, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term “polynucleotide” also includes DNAs or RNAs containing one or more modified bases and DNAs or RNAs with backbones modified for stability or for other reasons. “Modified” bases include, for example, tritylated bases and unusual bases such as inosine. A variety of modifications have been made to DNA and RNA; thus, “polynucleotide” embraces chemically, enzymatically or metabolically modified forms of polynucleotides as synthesized or as typically found in nature, as well as the chemical forms of DNA and RNA characteristic of viruses and cells. “Polynucleotide” also encompasses relatively short polynucleotides, often referred to as oligonucleotides. Such oligonucleotides could be isolated from nature or more typically, chemically synthesized.

The term “polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 amino acids represented by codons in the genetic code. “Polypeptides” include amino acid sequences modified either by natural processes, such as post-translational modification or processing, or by chemical modification techniques which are well known in the art. Such modifications are described in basic texts and in more detailed monographs, as well as in extensive research literature. Modifications can occur anywhere in a polypeptide, including the peptide backbone, the amino acid side-chains and the amino and/or carboxyl termini. It will be appreciated that the same type of modification may be present to the same extent or to varied extents at several sites in a given polypeptide. Also, a given polypeptide may contain many types of modifications. Polypeptides may be branched as a result of ubiquitination, and they may be cyclic, with or without branching. Disulfide bridges may form within or between polypeptide chains. Cyclic, branched and branched cyclic polypeptides may result from natural post-translational processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. See, for instance, PROTEINS—STRUCTURE AND MOLECULAR PROPERTIES, 2nd Ed., T. E. Creighton, W. H. Freeman and Company, New York, 1993 and Wold, F., Posttranslational Protein Modifications: Perspectives and Prospects, pgs. 1-12 in POSTTRANSLATIONAL COVALENT MODIFICATION OF PROTEINS, B. C. Johnson, Ed., Academic Press, New York, 1983; Seifter et al., “Analysis for protein modifications and nonprotein cofactors”, Meth Enzymol (1990) 182:626-646 and Rattan et al., “Protein Synthesis: Posttranslational Modifications and Aging”, Ann NY Acad Sci (1992) 663:48-62. In addition to these modifications and alterations of polypeptides, proteins may also associate with each other in various ways. Where used herein, “dimers” are an association of two proteins to form a single functional unit. “Homodimers” contain two identical subunits, while “heterodimers” contain two nonidentical subunits. “Multimers” contain two or more subunits per functional unit and may comprise identical and nonidentical polypeptide chains.

The term “substantially pure” refers to a preparation comprising at least 50-60% by weight the compound of interest (e.g., nucleic acid, oligonucleotide, protein, etc.). More preferably, the preparation comprises at least 75% by weight, and most preferably 90-99% by weight, the compound of interest. Purity is measured by methods appropriate for the compound of interest (e.g. chromatographic methods, agarose or polyacrylamide gel electrophoresis, HPLC analysis, and the like). Where used herein above the term “by weight” means the weight of the sample, exclusive of water and salts.

The term “substantially the same” refers to nucleic acid or amino acid sequences having sequence variation that do not materially affect the nature of the protein (i.e. the structure, stability characteristics, substrate specificity and/or biological activity of the protein). With particular reference to nucleic acid sequences, the term “substantially the same” is intended to refer to the coding region and to conserved sequences governing expression, and refers primarily to degenerate codons encoding the same amino acid, or alternate codons encoding conservative substitute amino acids in the encoded polypeptide. With reference to amino acid sequences, the term “substantially the same” refers generally to conservative substitutions and/or variations in regions of the polypeptide not involved in determination of structure or function.

The terms “percent identical” and “percent similar” are also used herein in comparisons among amino acid and nucleic acid sequences. When referring to amino acid sequences, “identity” or “percent identical” refers to the percent of the amino acids of the subject amino acid sequence that have been matched to identical amino acids in the compared amino acid sequence by a sequence analysis program. “Percent similar” refers to the percent of the amino acids of the subject amino acid sequence that have been matched to identical or conserved amino acids. Conserved amino acids are those which differ in structure but are similar in physical properties such that the exchange of one for another would not appreciably change the tertiary structure of the resulting protein. Conservative substitutions are defined in Taylor (1986, J. Theor. Biol. 119:205). When referring to nucleic acid molecules, “percent identical” refers to the percent of the nucleotides of the subject nucleic acid sequence that have been matched to identical nucleotides in the comparison sequence.

“Identity” and “similarity” can be readily calculated by known methods. Nucleic acid sequences and amino acid sequences can be compared using computer programs that align the similar sequences of the nucleic or amino acids thus define the differences. The Blastn and Blastp 2.0 programs provided by the National Center for Biotechnology Information (at http://www.ncbi.nlm.nih.govlblast/; Altschul et al., 1990, J Mol Biol 215:403-410) using a gapped alignment with default parameters, may be used to determine the level of identity and similarity between nucleic acid sequences and amino acid sequences.

With respect to single-stranded nucleic acid molecules, the term “specifically hybridizing” refers to the association between two single-stranded nucleic acid molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule, to the substantial exclusion of hybridization of the oligonucleotide with single-stranded nucleic acids of non-complementary sequence.

With respect to oligonucleotides, but not limited thereto, the term “specifically hybridizing” refers to the association between two single-stranded nucleotide molecules of sufficiently complementary sequence to permit such hybridization under pre-determined conditions generally used in the art (sometimes termed “substantially complementary”) In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence.

A “coding sequence” or “coding region” refers to a nucleic acid molecule having sequence information necessary to produce a gene product, when the sequence is expressed. A “coding sequence” may be determined indirectly from a known polypeptide sequence by understanding the genetic code. Since each amino acid is coded for by a codon containing three nucleotide bases, it is easy to ‘back-translate from a polypeptide sequence to a corresponding nucleotide sequence using a simple table of codon and their amino acid equivalents. Redundancy in the genetic code and “wobble” allow many possible “degenerate” sequences to encode the polypeptide of interest. A specific choice of a representative nucleotide sequence may be made on the basis of codon usage preference or codon bias, or degenerate sequences can be used for purposes where the ambiguity can be tolerated. Many of the commonly available molecular biology and/or molecular genetic computer packages provide a back-translation function. Other back-translation applications are available for public use or free download on the Internet.

Transcriptional and translational control sequences are DNA regulatory sequences, such as promoters, enhancers, polyadenylation signals, terminators, and the like, that provide for the expression of a coding sequence in a host cell.

The terms “promoter”, “promoter region” or “promoter sequence” refer generally to transcriptional regulatory regions of a gene, which may be found at the 5′ or 3′ side of the coding region, or within the coding region, or within introns. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase.

The term “operably linked” or “operably inserted” means that the regulatory sequences necessary for expression of the coding sequence are placed in a nucleic acid molecule in the appropriate positions relative to the coding sequence so as to enable expression of the coding sequence. This same definition is sometimes applied to the arrangement other transcription control elements (e.g. enhancers) in an expression vector.

A “vector” is a replicon, such as plasmid, phage, cosmid or virus, to which another nucleic acid segment may be operably inserted so as to bring about the replication or expression of the segment.

The term “nucleic acid construct” or “DNA construct” refers to genetic sequence used to transform cells or organisms. The term is sometimes used to refer to a coding sequence or sequences operably-linked to appropriate regulatory sequences and inserted into a vector. This term may be used interchangeably with the term “transforming DNA”. Such a nucleic acid construct may contain a coding sequence for a gene product of interest, along with a selectable marker gene and/or a reporter gene. The transforming DNA may be prepared according to standard protocols such as those set forth in “Current Protocols in Molecular Biology”, eds. Frederick M. Ausubel et al., John Wiley & Sons, 1999. Methods of transformation are specific to the kinds of cells transformed and are well known in the art.

The term “selectable marker gene” refers to a gene encoding a product that, when expressed, confers a selectable phenotype such as antibiotic resistance on a transformed cell.

The term “reporter gene” refers to a gene that encodes a product which is readily detectable by standard methods, either directly or indirectly.

A “heterologous” region of a nucleic acid construct is an identifiable segment (or segments) of the nucleic acid molecule within a larger molecule that is not found in association with the larger molecule in nature. Thus, when the heterologous region encodes a mammalian gene, the gene will usually be flanked by DNA that does not flank the mammalian genomic DNA in the genome of the source organism. In another example, a heterologous region is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein. The term “DNA construct”, as defined above, is also used to refer to a heterologous region, particularly one constructed for use in transformation of a cell.

A cell has been “transformed” or “transfected” by exogenous or heterologous DNA when such DNA has been introduced inside the cell. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations.

“Variant”, as the term is used herein, is a polynucleotide or polypeptide that differs from a reference polynucleotide or polypeptide respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one represented in the genetic code. A variant of a polynucleotide or polypeptide may be naturally occurring such as an allelic variant, or a single nucleotide polymorphism (SNP) or it may be a variant that is not known to occur naturally. Non-naturally occurring variants of polynucleotides and polypeptides may be made by mutagenesis techniques or by direct synthesis.

The term “antibodies” as used herein includes polyclonal and monoclonal antibodies, chimeric, single chain, and humanized antibodies, as well as F_{ab} fragments, including the products of an F_{ab} or other immunoglobulin expression library. With respect to the antibodies of the invention, the term, “immunologically specific” refers to antibodies that bind to one or more epitopes of a protein of interest, but which do not substantially recognize and bind other molecules in a sample containing a mixed population of antigenic biological molecules.

II. Description

Provided in accordance with the present invention is a green fluorescent protein (GFP), isolated from Renilla reniformis or synthesized to comprise a functionally equivalent amino acid sequence as that of the native Renilla reniformis GFP. Renilla GFP has several highly advantageous properties as compared with Aequorea victoria GFP, including an improved absorption spectrum, a higher molar extinction coefficient and improved stability.

GFP was purified from Renilla reniformis using previously described methods (Ward and Cormier, 1979, supra) . The GFP protein preparations were considered pure enough for protein sequencing when the ratio of absorbance at 498 nm to 280 nm was over 5.5. The purified polypeptide was fragmented by chemical and/or enzymatic means and the resulting overlapping fragments were subjected to HPLC, mass spectroscopy, and amino acid sequence analysis. Sequences of the fragments were aligned based on sequence overlaps to generate the polypeptide sequences set forth in SEQ ID NO: 1 and in SEQ ID NO: 46.

Referring to SEQ ID NO: 1, in certain embodiments, residues 124-127 are composed of the amino acid sequence Tyr-X₁-Gly-X₂, where X₁ is Lys or Arg and X₂ is Ser or Asn. In another preferred embodiment, when X₁ is Arg, X₂ is Asn or when X₁ is Lys, X₂ is Ser. In another preferred embodiment, residue 128 is a Lys, if the residue is not a Lys, then it is absent in other embodiments. In other preferred embodiments, residue 129 is Asp, Gly or Asn; residue 130 is Leu or Pro; residue 131 is Arg or Pro; and residue 132 is Glu, Arg, Leu, Ser or Asp. In another preferred embodiment, the residue at position 162 is a Cys, Trp or Thr, while in other preferred embodiments the residue is modified or a degradation product of Cys, Trp, or Thr. In another preferred embodiment, residues 217 and 218 are Thr or Glu and Thr or Gly respectively. In another preferred embodiment, the C-terminal portion of the protein extends beyond the proline residue 234, comprising the three amino acid sequence Glu-Trp-Val. In other embodiments the C-terminus contains other extensions or modifications, while in some embodiments such modifications are absent. In another embodiment, the N-terminal region of the protein is blocked or modified by one or more unusual or modified amino acids.

The Renilla GFP amino acid sequence of SEQ ID NO: 1 contains at residues 65-67, the chromophore characterized in Aequorea GFP. The Renilla sequence of this invention also contains an Arg residue at position 95 and a Glu at position 218. These two amino acids are present in all GFPs sequenced to date (numbered as residues 96 and 222, respectively, in Aequoria GFP) and have been postulated by Ward to be critical in productively interacting with the chromophore (Ward, 1998, In Green Fluorescent Protein: Properties, Applications and Protocols, pp 45-75, ed. M. Chalfie and S. Kain, Wiley-Liss). Because of the similarities in biological functions, physical properties, amino acid sequence and composition, the tertiary structure of Renilla GFP had been expected to be very similar to Aequorea GFP (Yang et al., 1996 supra).

The amino acid sequence set forth herein as SEQ ID NO: 46 is one preferred embodiment of the Renilla reniformis GFP sequence.

Due to the general unavailability of Renilla reniformis and the difficulty associated with purifying significant quantities of GFP from the organism itself, preferred methods of making the GFP of the present invention include: (1) synthesizing the polypeptide, using the amino acid sequence information set forth herein; and (2) back-translating the amino acid sequence to generate a nucleotide sequence, then synthesizing the nucleic acid and expressing it in an appropriate expression vector. In connection with this second method of making the GFP, and as discussed in greater detail below, a particularly preferred embodiment of back-translation employs codon preferences of the organism in which the GFP is desired to be expressed.

A GFP produced by the aforementioned methods and having the amino acid sequence of SEQ ID NO: 1, or that of SEQ ID NO: 46, is expected to possess the features of native Renilla GFP. Renilla GFP has excitation peaks at 470 nm and 498 nm, an emission peak at 509 nm and a region of low absorbance from 320-390 nm. The Renilla GFP also has a very high extinction coefficient, 133,000 at 498 nm. Additionally, this GFP is stable in 8 M urea, 6 M guanidine hydrochloride, 1% SDS and at high and low pH extremes

GFPs with amino acid residue variations, similar to those characterized in Aequorea, are very likely to have counterparts in Renilla; such mutations and variations will produce similar useful phenotypic changes in Renilla GFP. Mutants, including single nucleotide polymorphisms (SNPs) with these types of variations in amino acid sequence, are considered part of the present invention. Some of these types of variations are described in Ward (1998, supra), and in commonly-owned, co-pending U.S. patent application Ser. No. 60/104,563, all of which are incorporated by reference herein.

III. Preparation of Renilla reniformis GFP Proteins, Antibodies and Nucleic Acid Molecules

A. Synthesis of Renilla GFP Protein

The synthetic Renilla GFP protein of the present invention may be prepared by various synthetic methods of peptide synthesis via condensation of one or more amino acid residues, utilizing conventional peptide synthesis methods. Preferably, peptides are synthesized according to standard solid-phase methodologies, such as may be performed on an Applied Biosystems Model 430A peptide synthesizer (Applied Biosystems, Foster City, Calif.), according to manufacturer's instructions. Other methods of synthesizing peptides or peptidomimetics, either by solid phase methodologies or in liquid phase, are well known to those skilled in the art.

When solid-phase synthesis is utilized, the C-terminal amino acid is linked to an insoluble carrier that can produce a detachable bond by reacting with a carboxyl group in a C-terminal amino acid. One preferred insoluble carrier is p-hydroxymethylphenoxymethyl polystyrene (HMP) resin. Other useful resins include, but are not limited to, phenylacetamidomethyl (PAM) resins for synthesis of some N-methyl-containing peptides (this resin is used with the Boc method of solid phase synthesis) and MBHA (p-methylbenzhydrylamine) resins for producing peptides having C-terminal amide groups.

During the course of peptide synthesis, amino acid functional groups may be protected/deprotected as needed, using commonly-known protecting groups. For instance, side-chain functional groups consistent with Fmoc synthesis are protected as follows: arginine (2,2,5,7,8-pentamethylchroman-6-sulfonyl), asparagine (O-t-butyl ester), cysteine, glutamine and histidine (trityl), lysine (t-butyloxycarbonyl), serine and tyrosine (t-butyl). Modification utilizing alternative protecting groups for peptides and peptide derivatives will be apparent to those of skill in the art.

B. Production of Renilla GFP by Expression of a GFP-Encoding Nucleic Acid Molecule

The availability of amino acid sequence information, such as the sequence in SEQ ID NO: 1, or that in SEQ ID NO: 46, enables the preparation of a synthetic gene that can be used to synthesize the Renilla GFP protein via standard in vitro and in vivo expression systems. The sequence encoding Renilla GFP from isolated native nucleic acid molecules can be utilized as well. Alternately, an isolated nucleic acid that encodes the amino acid sequence of the invention can be prepared by oligonucleotide synthesis. In a preferred embodiment, codon usage tables are used to design a synthetic sequence that is particularly suited for a preferred organism. In a preferred embodiment, the codon usage table is derived from the organism in which the synthetic nucleic acid is expressed. For example, the codon usage for E. coli is used to design a DNA construct for expression of the Renilla GFP in E. coli. Organisms of interest include, but are not limited to, Renilla reniformis, Renilla kollikeri, other Renilla species, E. coli, yeast, insects plants, and mammals. In a preferred embodiment, preference is given to mammalian codon usage, for expression in mouse cells. In other preferred embodiments, codon usage for humans is used. GFP so expressed may find preferential use for example in certain diagnostic applications or in the field of experimental medicine. In a more preferred embodiment, a humanized GFP is designed with C-terminal His tags to facilitate purification after expression in a suitable cell expression system.

Synthetic oligonucleotides may be prepared by the phosphoramadite method employed in the Applied Biosystems 38A DNA Synthesizer or similar devices. The resultant oligonucleotide(s) may be purified according to methods known in the art, such as high performance liquid chromatography (HPLC). Long, double-stranded polynucleotides must be synthesized in stages, due to the size limitations inherent in current oligonucleotide synthetic methods. Thus, for example, a 1 kb double-stranded molecule may be synthesized as several smaller segments of appropriate complementarity. Complementary segments thus produced may be annealed such that each segment possesses appropriate cohesive termini for attachment of an adjacent segment. Adjacent segments may be ligated by annealing cohesive termini in the presence of DNA ligase to construct an entire 1.0 kb double-stranded molecule. A synthetic DNA molecule so constructed may then be cloned and amplified in an appropriate vector.

The availability of nucleic acids molecules encoding the Renilla GFP enables production of the protein using expression methods known in the art. According to a preferred embodiment, the protein may be produced by expression in a suitable expression system. For example, part or all of a DNA molecule, such as a DNA encoding the amino acid sequence of SEQ ID NO: 1 or SEQ ID NO: 46, may be inserted into a plasmid vector adapted for expression in a bacterial cell, such as E. coli, or a eukaryotic cell, such as Saccharomyces cerevisiae or other yeast. Such vectors comprise the regulatory elements necessary for expression of the DNA in the host cell, positioned in such a manner as to permit expression of the DNA in the host cell. Such regulatory elements required for expression include promoter sequences, transcription initiation sequences and, optionally, enhancer sequences. Appropriate expression systems include, but are not limited to: E. coli, the baculovirus system, Picia spp., yeast and Arabidopsis spp.

Alternatively, a cDNA or gene may be cloned into an appropriate in vitro transcription vector, such a pSP64 or pSP65 for in vitro transcription, followed by cell-free translation in a suitable cell-free translation system, such as wheat germ or rabbit reticulocytes. In vitro transcription and translation systems are commercially available, e.g., from Promega Biotech (Madison, Wis.) or BRL (Rockville, Md.).

The GFP produced by gene expression in vitro or in a recombinant procaryotic or eukaryotic system may be purified according to methods known in the art. In a preferred embodiment, a commercially available expression/secretion system can be used, whereby the recombinant protein is expressed and thereafter secreted from the host cell, to be easily purified from the surrounding medium. If expression/secretion vectors are not used, an alternative approach involves purifying the recombinant protein by affinity separation, such as by immunological interaction with antibodies that bind specifically to the recombinant protein or fusion proteins such as His tags. Such methods are commonly used by skilled practitioners. In addition, the unusual chemical stability of the Renilla GFP can be used to facilitate its purification. A mixture of expression products can be raised or lowered to a pH that denatures most other proteins, but leaves the stable GFP intact. The intact protein is then separated from the degraded or denatured proteins. Likewise, chaotropic agents such as 8 M urea or 6 M guanidine hydrochloride, or detergents such as 1% SDS (sodium lauryl sulfate) can be used to selectively denature proteins while leaving Renilla GFP intact.

The Renilla GFP of the invention, prepared by one of the aforementioned methods, may be analyzed according to standard procedures. For example, the protein may be subjected to amino acid composition or amino acid sequence analysis, according to known methods. The stability and biological activity of the synthetic protein may be determined according to standard methods by characterizing the spectral properties of the protein and comparing them to those of native Renilla GFP (see Ward et al., 1979, supra). The purity of the protein may be assessed by determining the ratio of 498 nm to 280 nm absorbance, with a pure preparation having a ratio of approximately 6.0. The protein may be quantified by standard methods well known in the art.

In addition, batches of Renilla GFP after analysis and determination of purity as in the above, can be used to make standardized GFP. Lack of proper standards forces most GFP assays to be strictly qualitative. The use of standardized GFP will allow great advances in using GFP in quantitative assays. Standardized GFP will allow simple calibration of instruments and calibration of assays, ensuring that quantitation and detection are optimized. Standardized GFP are enabled by the novel spectral properties of the proteins of this invention, and when used in combination with the assays of this invention, and/or in combination with the reduction in background or the increase of fluorescence signal to noise ratio enabled by the proteins and methods of this invention will further enable substantial improvements in quantitation accuracy and lowered detection limits. Such standards can also be made available as kits or as parts of kits for assays or for calibration of instruments used in fluorescence measurement.

C. Antibodies Immunologically Specific to Renilla GFP

The present invention also provides antibodies that are immunologically specific to the Renilla reniformis or R. kollikeri GFPs, or selected epitopes of the GFPs of the invention. Polyclonal antibodies may be prepared according to standard methods. In a preferred embodiment, monoclonal antibodies are prepared, which are immunologically specific to various epitopes of the protein. Monoclonal antibodies may be prepared according to general methods of Köhler and Milstein, following standard protocols. Polyclonal or monoclonal antibodies which are immunologically specific to the Renilla GFP can be utilized for identifying and purifying such proteins. For example, antibodies may be utilized for affinity separation of proteins with which they are immunologically specific or to quantify the protein. Antibodies may also be used to immunoprecipitate proteins from a sample containing a mixture of proteins and other biological molecules.

D. Isolation of Native Renilla GFP Nucleic Acid Molecules

Nucleic acid molecules encoding the Renilla GFP may be isolated from appropriate Renilla strains using methods well known in the art. However, the isolation of nucleic acids from Renilla is not trivial, inasmuch as R. reniformis appears to comprise many nucleases and other components that interfere with the isolation of intact DNA and RNA.

However, once an appropriate sample of mRNA or genomic DNA is obtained, a cDNA or genomic DNA library can be constructed using standard methods. Native nucleic acid sequences may be isolated by screening Renilla cDNA or genomic libraries with oligonucleotides designed to match the Renilla coding sequence of GFP. In positions of degeneracy, where more than one nucleic acid residue could be used to encode the appropriate amino acid residue, all the appropriate nucleic acids residues may be incorporated to create a mixed oligonucleotide population, or a neutral base such as inosine may be used. The strategy of oligonucleotide design is well known in the art (see also Sambrook et al., Molecular Cloning, 1989, Cold Spring Harbor Press, Cold Spring Harbor N.Y.).

Alternatively, PCR (polymerase chain reaction) primers may be designed by the above method to match the Renilla coding sequence of GFP, and these primers used to amplify the native nucleic acids from isolated Renilla cDNA or genomic DNA. In a preferred embodiment, a cDNA clone is isolated from Renilla reniformis. In another preferred embodiment, a genomic clone is isolated from Renilla reniformis. In a highly preferred embodiment, the cDNA or the genomic clone isolated contain sequences which encode a polypeptide substantially the same as the polypeptide of SEQ ID NO: 1 or that of SEQ ID NO: 46.

In accordance with the present invention, nucleic acids having the appropriate sequence homology with a Renilla GFP synthetic nucleic acid molecule may be identified by using hybridization and washing conditions of appropriate stringency. For example, hybridizations may be performed, according to the method of Sambrook et al. (1989, supra), using a hybridization solution comprising: 5× SSC, 5× Denhardt's reagent, 1.0% SDS, 100 μg/ml denatured, fragmented salmon sperm DNA, 0.05% sodium pyrophosphate and up to 50% formamide. Hybridization is carried out at 37-42° C. for at least six hours. Following hybridization, filters are washed as follows: (1) 5 minutes at room temperature in 2× SSC and 1% SDS; (2) 15 minutes at room temperature in 2× SSC and 0.1% SDS; (3) 30 min-1 h at 37 ° C in 1× SSC and 1% SDS; (4) 2 h at 42-65° C. in 1× SSC and 1% SDS, changing the solution every 30 minutes.

One common formula for calculating the stringency conditions required to achieve hybridization between nucleic acid molecules of a specified sequence homology (Sambrook et al., 1989, supra):

Tₘ=81.5° C.+16.6Log[Na+]+0.41(% G+C)−0.63(% formamide)−600/#bp in duplex

As an illustration of the above formula, using [N+]=[0.368] and 50% formamide, with GC content of 42% and an average probe size of 200 bases, the Tₘ is 57 ° C. The Tₘ of a DNA duplex decreases by 1-1.5° C. with every 1% decrease in homology. Thus, targets with greater than about 75% sequence identity would be observed using a hybridization temperature of 42° C.

The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tₘ of the of the hybrid. Wash conditions should be as stringent as possible for the degree of identity of the probe for the target. In general, wash conditions are selected to be approximately 12-20° C. below the Tₘ of the hybrid. In regards to the nucleic acids of the current invention, a moderate stringency hybridization is defined as hybridization in 6× SSC, 5× Denhardt's solution, 0.5% SDS and 100 μg/ml denatured salmon sperm DNA at 42° C., and wash in 2× SSC and 0.5% SDS at 55° C. for 15 minutes. A high stringency hybridization is defined as hybridization in 6× SSC, 5× Denhardt's solution, 0.5% SDS and 100 μg/ml denatured salmon sperm DNA at 42° C., and wash in 1× SSC and 0.5% SDS at 65° C. for 15 minutes. A very high stringency hybridization is defined as hybridization in 6× SSC, 5× Denhardt's solution, 0.5% SDS and 100 μg/ml denatured salmon sperm DNA at 42° C., and wash in 0.1× SSC and 0.5% SDS at 65° C. for 15 minutes.

Nucleic acids of the present invention may be maintained as DNA in any convenient cloning vector. In a preferred embodiment, clones are maintained in plasmid cloning/expression vector, such as pBluescript (Stratagene, La Jolla, Calif.), which is propagated in a suitable E. coli host cell.

Renilla GFP nucleic acid molecules of the invention include DNA, RNA, and fragments thereof which may be single- or double-stranded. Thus, this invention provides oligonucleotides (sense or antisense strands of DNA or RNA) having sequences capable of hybridizing with at least one sequence of a nucleic acid molecule encoding the protein of the present invention. Such oligonucleotides are useful as probes for detecting Renilla GFP genes or transcripts. In one preferred embodiment, oligonucleotides for use as probes or primers are based on rationally-selected amino acid sequences chosen from SEQ ID NO: 1 or SEQ ID NO: 46. In a more preferred embodiment, the amino acid sequence used to base the oligonucleotide sequence on corresponds to amino acids 101-155 of the protein in SEQ ID NO: 1 or SEQ ID NO: 46. In another preferred embodiment, the sequence of amino acids from positions 107-150 of SEQ ID NOS: 1 or 26 are used. In preferred embodiments, the amino acid sequence information is used to make degenerate oligonucleotide sequences as is commonly done by those skilled in the art. In other preferred embodiments, the degenerate oligonucleotides are used to screen cDNA libraries from Renilla spp, especially Renilla kollikeri. In yet other preferred embodiments, Halistaure spp, Phialidium spp and other marine organisms are screened.

IV. Uses of Renilla GFP nucleic acid molecules and Renilla GFP protein

Renilla GFP can be used in any application where existing GFP is currently being used, as well as in new applications enabled by the novel properties of Renilla GFP. The GFP protein, or nucleic acids encoding the GFP protein, is used as a marker of protein localization and/or gene expression. The GFP is used to particular advantage where the addition of exogenous substrates is impractical, as in applications involving living cells, high throughput screening, and large scale agricultural and environmental monitoring. This protein is successfully expressed in heterologous systems because the chromogenic hexapeptide of GFP cyclizes spontaneously without the need of cofactors or enzymes.

Renilla GFP offers several advantages over Aequorea GFP that expand its range of applications. The much higher extinction coefficient of Renilla GFP enables in vivo expression methods where Aequorea GFP is too weak to detect. Renilla GFP's transparent absorbance window between 320 nm and 390 run allows this GFP to be used in double-labeling experiments that are impossible with Aequorea GFP. Fluorescent probes whose excitation and emission spectra are suitable to be used as secondary probes with Renilla GFP include, but are not limited to DAPI. Noise subtraction (scatter and autofluorescence) can be accomplished more readily with Renilla reniformis GFP because the protein is transparent from 320 nm to 390 nm and from 525 nm to 700 nm. Such noise subtraction is extremely beneficial in facilitating the fluorometric monitoring of turbid cell suspensions (as in live cell promoter-driven HTS systems) or in remote sensing applications in agricultural or environmental monitoring, such as monitoring crop development or soil conditions. The high chemical stability of GFP in general, and Renilla GFP in particular, allows it to be used to advantage in assay kits and other applications that involve biochemical manipulations and/or long term storage.

The GFP can be detected in these methods in several ways. As with Aequorea GFP, Renilla GFP can most advantageously be detected by using its unique fluorescent properties. Any of the general techniques for detecting Aequorea GFP can also be used for Renilla GFP as long as the unique characteristics of the Renilla GFP excitation spectra are taken into consideration. Renilla GFP can also be detected using any methods applicable to general protein detection, for example the use of antibodies specific to Renilla GFP. Methods for both of these approaches are well known in the art.

Because GFP is part of a larger system of fluorescence, it has the potential to be combined with the other components of the system to advantage. Luciferin and the luciferin-binding protein from Renilla can be used with Renilla GFP to change the excitation profile of GFP. The need for a close association of the two proteins for energy transfer can be used to test for the physical proximity of proteins to which they are fused in vivo.

Renilla GFP is particular well suited for pairing with Aequorea GFP for fluorescence resonance energy transfer (FRET) measurements. Intracellular and extracellular reporting by FRET may be accomplished by coupling a blue-emitting Tyr66 variant of Aequorea Victoria GFP (Y66H, Y66W, Y66F or the equivalent) to a green-emitting Renilla reniformis GFP. The interspecies (Aequorea-Renilla) FRET pairing is preferable to an intraspecies pairing (i.e. coupling an Aequorea blue-emitting variant to an Aequorea green- or yellow-emitting variant). The main reason for choosing an interspecies FRET pair is that all variants of Aequorea GFP self-associate to form reversible dimers (homodimers and heterodimers) (Barbieri et al., in 11th International Symposium on Bioluminescence and Chemiluminescence Symposium Proceedings, 2000). Thus, when two color variants of Aequorea GFP are used together in FRET determinations (as with two-hybrid energy transfer assays, in vivo), it may be impossible to determine whether the targeted proteins are drawing together the two color variants of Aequorea GFP to form an energy transfer pair or whether the self-association of the two Aequorea GFP variants is producing a false positive signal that has nothing to do with protein-protein self-association of the targeted cellular proteins.

Additionally, Renilla GFP is better suited than Aequorea GFP for fluorimetric assays. There is no wavelength from 250 nm through 520 nm that does not excite Aequorea GFP to fluoresce. There is no transparent window in the Aequorea GFP excitation spectrum over this range. Renilla GFP, however, does have a transparent excitation window that extends from 320 nm to 390 nm. This extended region of transparency (found in Renilla GFP but not in Aequorea GFP) provides a mechanism for significant noise reduction in Renilla GFP-based fluorimetric assays (microtiter plates and other high throughput screening devices). This noise reduction (or signal-to-noise enhancement) can be accomplished by employing polychromatic excitation optics in the fluorimetric detector. Thus, by exciting at 365 nm, 488 nm and 546 nm, for example, scatter and autofluorescence stimulated by 365 nm excitation and/or by 546 nm excitation can be eliminated from the true GFP fluorescence excited at 488 nm. In some cell-based fluorimetric assays, polychromatic excitation of this sort could result in a 1000-fold improvement in signal-to-noise ratio, when comparing an Aequorea-based assay with a Renilla-based assay.

A. GFP Nucleic Acids

Green Fluorescent Protein nucleic acids may be used for a variety of purposes in accordance with the present invention. DNA, RNA, or fragments thereof may be used as probes to detect the presence of and/or expression GFP genes. Methods in which GFP nucleic acids may be utilized as probes for such assays include, but are not limited to: (1) in situ hybridization; (2) Southern hybridization (3) Northern hybridization; and (4) assorted amplification reactions such as polymerase chain reactions (PCR)

The GFP nucleic acids of the invention may also be utilized as probes to identify related genes from other Renilla species or from other anthozoan coelenterates. As is well known in the art, hybridization stringencies may be adjusted to allow hybridization of nucleic acid probes with complementary sequences of varying degrees of homology.

As described above, GFP nucleic acids may be used to advantage to produce large quantities of substantially pure Renilla GFP, or selected portions or epitopes thereof. The protein is thereafter used for various commercial purposes, as described below. In a preferred embodiment of the invention, large amounts of the recombinant Renilla GFP can be made by in vitro or in vivo expression systems.

The GFP coding sequence can also be used as a reporter protein in transgenic cells or organisms. In a preferred embodiment of the invention, a Renilla GFP coding sequence is operably fused to the coding sequence of a protein of interest, an appropriate promoter region and termination region, and transformed into a cell. In this manner, the localization of a protein of interest can be determined in vivo, using the fluorescent properties of the fused GFP protein. Fusions of this nature can localize proteins to specific structures of the cell, such as the cytoskeleton, plasma membrane, nucleus, mitochondria, secretory pathway, and can also be used to study, in vivo, dynamic changes in the distribution and/or turnover of proteins within the cell, or within an organism. Such fusion proteins can also be used as an indicator of protein-protein interactions: the interaction a GFP fusion protein and a fusion protein comprised of a second fluorescent protein, i.e. anthozoan luciferase, may be detected by the resonance transfer of energy from one fluorescent molecule to the other.

In another preferred embodiment, the GFP coding sequence is operably-linked to a promoter region of interest and termination sequences, and used as a reporter gene to transform a cell. These transgenic cells can be used to advantage to study the regulation of the promoter region of interest in vivo or to trace cell lineage. Such studies are expected to reveal many subtle aspects of promoter regulation due to the exquisite sensitivity of these GFP assays using Renilla GFP. In a particularly preferred embodiment, GFP nucleic acids are used to construct specific cell lines for cell-based diagnostics. Screening for compounds that regulate specific promoters can be accomplished using custom-designed cell lines combined with robot-compatible methodology. This embodiment is particularly applicable for screening drugs, organic chemicals, pesticides, mutagens, carcinogens and teratogens. In another preferred embodiment, Renilla reniformis GFP is used in agricultural or environmental applications as a reporter of plant stress, soil conditions, or crop development using remote fluorescence detecting technologies.

B. Renilla GFP

The GFP protein can be used as a label in many in vitro applications currently used. Purified GFP can be covalently linked to other proteins by methods well known in the art, and used as a marker protein. The purified GFP protein can be covalently linked to a protein of interest in order to determine localization. In particularly preferred embodiments, a linker of 4 to 20 amino acids is used to separate GFP from the desired protein. This application may be used in living cells by micro-injecting the linked proteins. The GFP may also be linked chemically or genetically to antibodies and used thus for example in localization of antigens in fixed and sectioned cells, or in other immunological applications (e.g. dot blotting, western blotting) known to those skilled in the art. In the case of Renilla GFP-antibody fusion proteins, GFP may be used in numerous immunological assays where a heavy chain polyclonal antibody fused to Renilla GFP at the C-terminus of the heavy chain may preclude the need for a secondary fluorometrically-tagged antibody.

The GFP may be linked to purified cellular proteins and used to identify binding proteins and nucleic acids in assays in vitro, using methods well known in the art.

The GFP protein can also be linked to nucleic acids and used to advantage. Applications for nucleic acid-linked GFP include, but are not limited, to FISH (fluorescent in situ hybridization), and labeling probes in standard methods utilizing nucleic acid hybridization.

The following examples are provided to describe the invention in greater detail. They are intended to illustrate, not to limit, the invention.

### EXAMPLE 1

### Cloning and Characterization of a cDNA from Renilla reniformis: Artificial Gene Construction

Construction of an artificial gene encoding the R. reniformis GFP was undertaken according to method of Stemmer et al; 1995 in “Single-step assembly of a gene and entire plasmid from large numbers of oligodeoxyribonucleotides” in GENE 164: 49-53 (1995).

Determination of a Nucleotide Sequence Encoding GFP from R. reniformis

The amino acid sequence of GFP from Renilla reniformis, SEQ ID NO: 1, was back-translated to its corresponding nucleotide sequence as set forth in SEQ ID NO: 2. A codon usage preference for bacteria/E. coli was specified. Additionally, several minor changes were made in nonessential sequence to allow the introduction of two restriction endonuclease cleavage sites, and to encode a Histidine tag at the carboxy terminus to allow for easy of purification of the expressed protein. A cleavage site for NdeI (CATATG) was added immediately upstream of the AUG codon for the N-terminal methionine, and a XhoI cleavage site (CTCGAG) was engineered at the carboxyl terminus. Several additional amino acids were added to the C-terminus including a polyhistidine tag. GFP is particularly amenable to fusion with other proteins or short polypeptides and these in no way interfere with the desirable properties or expression of the protein. The complete amino acid sequence encoded by the open reading frame of the modified, back-translated nucleotide of SEQ ID NO: 2 is set forth as the amino acid sequence SEQ ID NO: 3.

Gene Assembly

Strategic Selection of Synthetic Oligonucleotides

A series of oligonucleotides corresponding to the each of the complementary strands of the back-translated nucleotide sequence were prepared according to the strategy outlined by Stemmer et al (1995, supra). According to the strategy, a series of consecutive oligonucleotides, which in their entirety comprise the full length of the back-translated nucleotide sequence, were generated. The nineteen oligonucleotides, SEQ ID NOs: 4 through 22, hereinafter the upper primers, were each 40-mer oligonucleotides corresponding to the first (upper) strand of the back-translated sequence provided in SEQ ID NO: 2. The nineteen oligonucleotides SEQ ID NOs: 23 through 41, hereinafter the lower primers, were each 40-mer oligonucleotides corresponding to the second (lower) strand of the back-translated sequence (i.e. the complement of SEQ ID NO: 2). Oligonucleotides 4-41 were purchased from Integrated DNA Technologies (IDT, Coralville, Iowa).

The corresponding nucleotides for construction of an artificial gene encoding the amino acid sequence as set forth in SEQ ID NO: 46 are provided as SEQ ID NO's: 47-84. The experiments are analogous to those described herein, using these primers instead.

DNA Polymerase Helps to Create the Full-Length Gene

Each oligonucleotide is constructed to have a 20-nucleotide “overlap” of complementarity with its neighbor oligonucleotides on the opposing strand. Under proper conditions of stringency, the set of consecutive oligonucleotides will hybridize with its neighbors. The set of upper and lower primers are mixed in equal concentration under proper conditions and Taq DNA polymerase is added. Under PCR conditions, repeated cycles of DNA polymerase action on the hybridized, aligned and overlapping oligonucleotides eventually yield the full-length properly assembled gene.

Gene Amplification

An aliquont of the reaction mixture from the Gene Assembly step containing the full-length product above is then amplified via PCR with Taq DNA polymerase, in the presence of dNTPs, and, as primers, the oligonucleotides corresponding to the 5′ ends of both the upper and lower strands of the back-translated SEQ ID No: 1.

The product of the gene assembly step is purified and separated by electrophoresis on 1% agarose gel. The purified product is digested with NdeI and Xhol restriction endonucleases; the plasmid pET24A (Novagene, Madison, Wis.) is likewise digested with the same enzymes. The fragment and the plasmid are ligated, and transformed into E. coli.

Characterization of the GFP Clone

Transformants containing the plasmid are grown and plasmid DNA is obtained. The clone is sequenced to verify the proper full-length clone has been selected. The GFP clone is inserted in frame with the His tag of the expression plasmid. The plasmid is then used in expression experiments, to generate quantities of the cloned GFP protein. The protein is readily purified and the His tag facilitates purification via immobilized metal affinity chromatography, which provides great advantage in rapid purification.

The purified protein can be used to generate batches of standardized cloned GFP with reproducible spectral properties, and is used for calibration of instruments or assays.

### EXAMPLE 2

Cloning of a cDNA encoding GFP from Renilla reniformis

The cloning of an intact, full-length cDNA encoding GFP from Renilla reniformis was undertaken according to the method of Matz et al. (Nature Biotechnology 17: 969-973, 1999).

Isolation of mRNA from R. reniformis

The total RNA from the sea pansy, R. reniformis, was isolated using a Stratagene RNA isolation kit. Subsequently, mRNA was isolated from the total RNA with the magnetic PolyA Tract mRNA Isolation System III (Promega).

Back-Translation Protein Sequence and Design of Primers

The amino acid sequence of the Renilla GFP, as set forth in SEQ ID NO: 1, was used to generate a back-translated nucleotide sequence as set forth in SEQ ID NO: 2. The nucleotide sequence was selected for codon usage bias of E. coli. The sequence in this back-translated sequence was used to design two oligonucleotide primers, GSP1 and GSP2, respectively SEQ ID Nos: 44 and 45. The first primer GSP1 was used in conjunction with SMART PCR (below) to obtain a nucleotide fragment corresponding to the C-terminus. Nested PCR is performed to obtain sequence towards the N-terminus.

SMART PCR cDNA Synthesis and Amplification

A SMART PCR cDNA synthesis Kit (Clontech) was used for the first strand cDNA synthesis from polyA mRNA. The manufacturer's protocol (SMART PCR cDNA Synthesis Kit User Manual PT3041-1, Published Apr. 27, 1999 by Clontech which is herein incorporated by reference in its entirety), except that the TN3 primer (5′-CGCAGTCGACCG(T)13), SEQ ID NO: 42, was used instead of the kit's CDS primer.

The cDNA population was amplified by PCR using the primers TS (5′-AAGCAGTGGTATCAACGCAGAGT), SEQ ID NO: 43 and TN3, SEQ ID NO: 42 (and above), each at 0.1 μm. The cDNA was diluted 20-fold with water and 1 μl of this was used in the PCR reaction as described in the kit instructions.

Modified 3′ RACE of the GFP

A gene-specific primer, designated GSP1was designed. The primer was purchased from IDT (IA) and had the sequence set forth in SEQ ID NO: 44. The first of two PCR steps used the GSP1 and TN3 primers. An aliquot of 1 μl of a 20-fold diluted cDNA mixture of the amplified cDNA was 1 5 added to a reaction mixture containing Advantage KlenTaq Polymerase mix (Clontech), the manufacturer's 1× reaction buffer, 200 μM dNTPs (Gibco BRL), 0.3 μM GSP! and 0.1 μM TN3 primer in a total volume of 20 μl. Cycling was performed in a Perkin Elmer Gene Amp PCR System 2400. PCR conditions included: 1 cycle of: 95 C. for 10 s, 55 C. for 1min, 72 C. for 40 s and 24 cycles of 95 C. for 10 s, 62 C. for 30 s and 72 C. for 40 s.

The reaction products were then diluted 20-fold and 1 μl of the diluted mixture are added to a second PCR which contained Advantage KlenTaq Polymerase mix (Clontech), the manufacturer's 1× reaction mix, 200 μM dNTPs (Gibco BRL), 0.3 μM primer GSP2 (SEQ ID NO: 45), and 0.1 μM TN3 primer in a total volume of 20 μl. The PCR conditions were as follows: 1 cycle of 95 C. for 10 s, 55 C. for 1 min, 72 C. for 40 s; then 13 cycles of 95 C. for 10 s, 62 C. for 30 s and 72 C. for 40 s.

The 5′ end of the cDNA is obtained by following the method of Modified 5′ RACE PCR. The 3′ fragment is isolated from the PCR and sequenced. A 3′ gene-specific primer is designed to function in PCR with a 5′ primer. In other words, the cloned 3′ end of the cDNA is combined with a cloned 5′ end of the cDNA obtained, both fragments obtained via Modified RACE PCR. The fragments are aligned, ligated together, and cloned as a full-length cDNA.

Characterization of the Full-Length cDNA

The fill-length cDNA is sequenced to verify the integrity of the clone. The deduced amino acid sequence of the open reading frame is also compared with the amino acid sequences in SEQ ID NO: 1. After sequencing, the full-length PCR fragment is inserted into the expression vector pET24A (Novagene). The protein is then expressed in large quantity in an E. coli expression system.

### EXAMPLE 3

Purification and Characterization of GFP from Renilla kollikeri

Purification

Starting with approximately 2 kg of sea pansy (Renilla kollikeri), the method of Gonzalez & Ward for large-scale purification of GFP from E. coli was followed (Daniel G Gonzalez and William W Ward; “Large scale Purification of Recombinant Green Fluorescent Protein from Escherichia coli” pp 212-223 Methods in Enzymology; Volume 305; Bioluminescence and Chemiluminescence; Part C; edited by Miriam M. Ziegler and Thomas 0 Baldwin; Academic Press; 2000).

Characterization

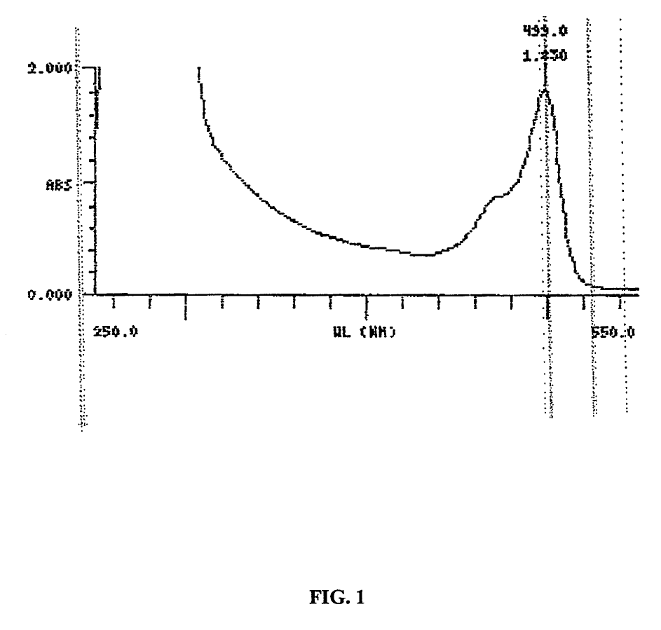

The purification yielded about 1 mg of purified GFP. The absorbance spectrum of the GFP from R. kollikeri was identical with that of R. reniformis, including the near-transparent window of absorption between 320-390 nm (FIG. 1). The behavior of the protein throughout the purification scheme was substantially similar to that of the R. reniformis GFP. This is evidence of the similarity of physical, chemical and biochemical properties between the two GFPs.

Determination of Amino Acid Sequence

Samples of the purified GFP are chemically and/or enzymatically digested to generate fragments. These fragments are subjected to HPLC and mass spectroscopy, and the characterized and isolated fragments are then subjected to sequencing via automated Edman degradation. The final sequence of the GFP is assembled by alignment of overlapping sequences of the fragments. Comparisons are made to the sequence of the completed R. reniformis to speed analysis of the completed fragment data. The complete sequence is substantially identical to that of R. reniformis. Certain conservative amino acid substitution are acceptable in nonessential areas of the protein (i.e. those not critical for the function of the chromophore, and those not critical to maintaining the tertiary structure of the folded protein).

Cloning R. kollikeri cDNA

In addition to the protein sequence, clones are obtained from R. kollikeri. The cDNA from R. reniformis is used as a probe to identify genomic and/or cDNA clones. Isolated R. kollikeri polyA mRNA is used as a source of full-length MRNA corresponding to the GFP. Standard techniques are used to prepare a cDNA library containing the desired sequence. The cDNA is placed into a vector appropriate for expression in the desired organism. Alternatively, a series of oligonucleotides corresponding to each strand of the full length of a back-translation of the R. kollikeri GFP amino acid sequence is prepared. The overlapping oligonucleotides are annealed and ligated to create a synthetic GFP gene. Strategic placement of proper cloning sites (e.g. restriction endonuclease cleavage sites) allows the synthetic GFP gene to be placed into a proper cloning vector. Sequencing of the cloned nucleic acid is performed to verify that the clone is correct and of full length. The selected vector is appropriate for expression in a desired system, for example, pET24A (Novagene) for expression in E. coli. The cDNA is optimized for expression in the desired organism by adapting the sequence to the codon usage preferences of the desired organism. Large-scale preparation or commercial production of the GFP is enabled by the availability of the cloned GFP and an appropriate expression system.

The present invention is not limited to the embodiments described and exemplified above, but is capable of variation and modification without departure from the scope of the appended claims.

We claim:

1. An isolated polypeptide having an amino acid sequence that confers upon the polypeptide physical and biochemical properties of a green fluorescent protein (GFP) from Renilla reniformis or Renilla kollikeri.

2. The isolated polypeptide of claim 1, further comprising a GFP chromophore.

3. The isolated polypeptide of claim 1, further comprising excitation spectrum peaks at 470 nm and 498 nm.

4. The isolated polypeptide of claim 1, further comprising a region of low absorbance of light energy in the range from 320 nm to 390 nm.

5. A variant of the isolated polypeptide of claim 1, having an excitation or emission spectra that is different from the excitation or emission spectra of a native GFP from Renilla reniformis or Renilla kollikeri.

6. The isolated polypeptide sequence of claim 1 comprising an amino acid sequence substantially the same as the Renilla reniformis sequence set forth in SEQ ID NO: 46.

7. An isolated GFP comprising an amino acid sequence substantially the same as a sequence selected from the group consisting of SEQ ID NO: 1, SEQ ID NO: 3, and SEQ ID NO: 46

8. The isolated GFP of claim 7 which includes a GFP chromophore.

9. The isolated GFP of claim 7 further comprising excitation and emission spectra of a Renilla GFP.

10. The isolated GFP of claim 9 which has an extinction coefficient equal to or greater than 70,000 L mol⁻¹ cm⁻¹ and a quantum yield of at least 0.5.

11. A variant of the isolated GFP of claim 7, having an excitation or emission spectra that is different from the excitation or emission spectra of a native GFP from Renilla reniformis or Renilla kollikeri.

12. An isolated or synthesized nucleic acid molecule which encodes the polypeptide of claim 1.

13. The nucleic acid of claim 12 wherein the sequence is substantially the same as the sequence set forth in SEQ ID 2.

14. The nucleic acid molecule of claim 12 further comprising sequence modifications selected from the group consisting of: adding or removing one or more restriction endonuclease cleavage sites, changing codon usage to optimize the sequence for expression in a selected organism, adding or removing one or more amino acids, and site-directed mutagenesis changes of one or more amino acids.

15. The nucleic acid molecule of claim 12, further comprising a sequence optimized for expression in an organism selected from the group consisting of bacteria, yeast, insects, plants and mammals.

16. An isolated nucleic acid molecule which encodes the polypeptide of claim 7.

17. Isolated antibodies of which specifically recognize and bind antigenic epitopes of Renilla GFP.

18. The isolate antibodies of claim 17 which specifically recognize and bind antigenic epitopes present in the polypeptide having the amino acid sequence set forth in SEQ ID NO: 46.

19. An antibody-GFP complex comprising noncovalent interaction between an antibody specific for Renilla GFP and the GFP recognized by said antibody.

20. A fusion protein comprising an antibody, or functional portion thereof, and a GFP.

21. A GFP standard comprising a composition of Renilla GFP with known physical, biochemical and biophysical properties.

22. The GFP standard of claim 21 wherein one or more of the extinction coefficient, quantum yield or other useful biophysical or spectral properties are predetermined.

23. The GFP standard of claim 22 used as a standard for calibration of instruments.

24. The GFP standard of claim 23 wherein the instrument is selected from the group consisting of: high-throughput screening monitors, fluorometers, fluorescence microscopes, fluorescence detectors, fluorescence activated cell sorters, flow cells, flow monitors, fluorescence spectrometers, fluorescence polarization instruments, x-ray fluorescence instruments, fluorescence imaging instruments, ratio fluorescence instruments, spectrofluorometers, fluorescence scanners, fluorescence-based microplate readers, fluorescence-based nucleic acid sequencing systems, laser- and laser diode-based fluorescence instruments, and charge-coupled device (CCD)-based fluorescence instruments.

25. A method of calibrating fluorescence-based biological assays with the GFP standard of claim 21, comprising one or more of the steps of:
a) adjusting a fluorescence reading instrument with a known amount of the GFP standard;
b) creating standard curves with the GFP standard, according to the conditions of the biological assay;
c) maintaining the instrument in proper calibration by checking periodically with the GFP standard;
d) comparing each assay or batch of assays performed with assay standard curve;
e) referring to the assay standard curve for accurate quantitation of the assay; and
f) including internal controls with each assay or batch of assays by adding a known amount of the GFP standard to an assay sample.

26. A kit for the calibration of fluorescence-based instruments and assays comprising:
a) the standard GFP of claim 21, and optionally, one or more of;
b) a series of concentrations of the GFP standard;
c) a certificate of quality control indicating batch and control numbers, concentrations of the standards and biophysical data about the standards; and
d) instructions for use of the kit to calibrate fluorescence-based instruments and biological assays.

27. An oligonucleotide for use as a primer or in screening or cloning new GFP-related molecules, comprising a nucleotide sequence derived from a nucleic acid molecule encoding an amino acid sequence selected from the group consisting of SEQ ID NO: 46 and SEQ ID NO: 1.

28. The oligonucleotide of claim 27 wherein the nucleotide sequence encodes amino acids 101-155 of SEQ ID NO: 1.

29. The oligonucleotide of claim 27 wherein the nucleotide sequence encodes the amino acids 107-150 of SEQ ID NO: 1.

30. An oligonucleotide for use as a primer or in screening for new GFP-related molecules comprising a nucleotide sequence derived from a portion of the nucleotide sequence set forth as SEQ ID NO: 2.

31. A method for reducing background noise and optimizing signal in fluorescence-based biological assays comprising one or more of the steps of:
a) using a GFP with a low absorbance window at one or more points in the spectrum, and high absorption and emission at other points in the spectrum;
b) using polychromatic filters to ensure that light of the proper wave lengths can be selected for the assay;
c) determining one or more optimum wavelengths for excitation and emission measurement based on the maximum light emitted from the sample versus the lowest amount of quenching, interference and nonspecific absorption from assay components; and
d) using a standard GFP for comparison and to determine loss of signal, quenching and energy transfer efficiency.
